(19) United States
(12) Patent Application Publication
(10) Pub. No.: US 2015/0148696 A1
(43) Pub. Date: May 28, 2015

(54) Electrocardiogram Pace Pulse Detection and Analysis

(71) Applicants: Carolyn Anjali Lall, Woburn, MA (US); Yu Chen, Andover, MA (US)

(72) Inventors: Carolyn Anjali Lall, Woburn, MA (US); Yu Chen, Andover, MA (US)

(73) Assignee: Draeger Medical Systems, Inc., Andover, MA (US)

(21) Appl. No.: 14/397,162

(22) PCT Filed: Apr. 27, 2012
(86) PCT No.: PCT/US2012/035647

§ 371 Date: Oct. 24, 2014

(51) Int. Cl. A61B 5/00; A61B 5/044; A61B 5/04; A61N 1/37; A61B 5/0452

(52) U.S. Cl. CPC A61B 5/7217; A61N 1/3706; A61B 5/04525; A61B 5/04017; A61B 5/044; A61B 5/0006
USPC 600/510

(57) ABSTRACT

Data is received characterizing an electrocardiogram (ECG) measured from a patient having a pacemaker. The pacemaker can intermittently provide a pace to a heart of the patient. By comparing at least a portion of the received data to a template a pace pulse can be detected from at least a portion of the received data. The template comprises one or more pace pulses measured from the patient. The template is modified, at least in part, by the detected pace pulse to update the template. Data characterizing the detected pace pulse is provided. Related apparatus, systems, techniques and articles are also described.

### TECHNICAL FIELD

The subject matter described herein relates to techniques, methods, systems and articles for improved electrocardiogram analysis. Particularly, improved electrocardiogram (ECG) pace pulse detection is provided.

### BACKGROUND

A pacemaker is a medical device that uses electrical impulses, delivered by electrodes contacting the heart muscles, to regulate the beating of the heart. The primary purpose of a pacemaker is to maintain an adequate heart rate, either because the heart's native pacemaker is not functioning properly, or there is a block in the heart's electrical conduction system. Some devices combine a pacemaker and defibrillator in a single implantable device. Other devices have multiple electrodes stimulating differing positions within the heart to improve synchronization of the chambers of the heart. A pacemaker can also be an implanted cardiac resynchronization device (CRD) and can be combined with or include an implantable cardioverter-defibrillator (ICD). A pacemaker can also be temporary, where the leads in contact with the heart are implanted, but the remaining portion of the device is outside the patient's body.

The 12-lead resting electrocardiogram (ECG) continues to play a key role in pacemaker follow-up and patient monitoring but automated analysis in patients with pacemakers is considered difficult due to pacemaker complexity and variability among device models. Modern ECG monitors are able to trigger alarms in response to abnormal heart characteristics and other cardiac events. If a patient being monitored has a pacemaker, the signal from the pacemaker can cause artifacts in the ECG signal that can trigger false alarms and/or can prevent the proper triggering of an alarm.

FIGS. 1A-C are a series of plots illustrating the effect a pacemaker can have on an ECG signal. For example, on plot 100, the pace signal can manifest as a square wave with rising edge 110 and falling edge 120. The pace signal can have a pace tail 130, which occurs after the square wave. The pace signal can manifest with many variation such as a negative square signal, slowly rising and/or falling edges (e.g. not a square wave), etc. Plots 140 and 150 are additional examples of pace signals.

According to the Association for the Advancement of Medical Instrumentation (AAMI), pacemaker pulses have the following characteristics at the body surface: pulse amplitude of 2-700 mV, pulse width of 0.1-2 ms and slew rate of 10 percent of pulse width or 100 us whichever is smaller. However, with improvements in pacemaker technology such as bipolar pacing, bi-ventricular pacing and automatic capture algorithms, solely meeting the AAMI standards, particularly the amplitude requirement, is no longer sufficient to effectively monitor patients with implantable pacemakers.

A pace pulse can be delivered by a pacemaker to different areas of the heart. For example, a pace pulse can be delivered to the right atrium (RA), the right ventricle (RV) or the left ventricle (LV). There are a variety of pace pulse shapes possible due to, for example: device manufacturer, device model, pacemaker lead model, pacemaker lead location, ECG electrode placement, device programming, etc. However, in most clinical situations these factors are static. Thus pace pulse morphology between paces delivered to the same area of the heart is consistent. For example, while the RA pacing signal morphology for patient A in ECG lead I will be different from the RV pacing pulse morphology, the RA pacing signal morphology will be consistent between RA pace pulses (and RV pace pulse morphology will be consistent between RV pace pulses). Moreover, the morphology of noise signals in patient A and ECG lead I will be more variable than the RA or RV pacing pulses.

FIG. 2 shows two plots 200 and 202 illustrating, for a relatively short period of time, the relatively consistent morphology of a pace pulse for a patient when compared to the variable morphology of noise signals measured. The set of pace pulses 204 show consistent morphology (i.e. they look similar and are strongly correlated). Noise pulses 208 show variable morphology (i.e. they do not look similar and do not appear to be strongly correlated).

Failure to accurately detect pacemaker pulses and remove the pace pulse artifact from the ECG waveform can result in inaccurate heart rate measurements, false arrhythmia calls and missed asystole calls. Therefore, it would be beneficial for an ECG monitor to accurately detect the occurrence of a pacemaker providing a pace to the patient's heart thereby reducing false alarms and missed alarms.

### SUMMARY

In one aspect, the subject matter disclosed herein describes receiving data characterizing an electrocardiogram (ECG) measured from a patient having a pacemaker. The pacemaker can intermittently provide a pace to a heart of the patient. By comparing at least a portion of the received data to a template a pace pulse can be detected from at least a portion of the received data. The template comprises one or more pace pulses measured from the patient. The template is modified, at least in part, by the detected pace pulse to update the template. Data characterizing the detected pace pulse is provided.

In another aspect, potential pace data is selected from at least a portion of the received data and detection is performed on the potential pace data.

One or more of the following features may also be included. The received data can be blanked for a blanking period to remove the detected pace pulse from the received data. The length of the blanking period can be determined by at least one characteristic of the detected pace pulse, the at least one characteristic can be one or more of a pace pulse width and a pace pulse amplitude. The detected pace signal can be further classified. The detected pace signal can be classified as an atrial pace pulse, right ventricular pace pulse or left ventricular pace pulse. The occurrence of the classified detected pace pulse can be calculated. The received data can be processed. The processing can include removing a baseline variation using a filter. The pace pulse can be detected when the amplitudes of the at least a portion of received data and the template differ by less than about 25%. The comparison of the at least a portion of the received data to the template can be calculated according to a correlation between the at least a portion of received data and a template. The comparison of the at least a portion of the received data to the template can be calculated according to:

$$r = \frac{{\sum }_{i = 1}^{n} ( {X}_{i} - \overline{X} ) ( {Y}_{i} - \overline{Y} )}{\sqrt{{\sum }_{i = 1}^{n} {( {X}_{i} - \overline{X} )}^{2}} \sqrt{{\sum }_{i = 1}^{n} {( {Y}_{i} - \overline{Y} )}^{2}}} .$$

The received data can include data from four or more leads. The at least a portion of received data can correspond to a subset of the ECG leads having the highest signal to noise ratio when compared to all leads. Providing the data can include one or more of persisting the data, displaying the data, or transmitting the data to a remote module or computing system. The template can be a dynamic template.

Articles of manufacture are also described that comprise computer executable instructions permanently stored (e.g., non-transitorily stored, etc.) on computer readable media, which, when executed by a computer, causes the computer to perform operations herein. Similarly, computer systems are also described that can include a processor and a memory coupled to the processor. The memory can temporarily or permanently store one or more programs that cause the processor to perform one or more of the operations described herein. In addition, methods can be implemented by one or more data processors either within a single computing system or distributed among two or more computing systems.

The subject matter described herein provides many advantages. For example, the current subject matter enables pacemaker pulse detection that can accurately identify the location of pacemaker pulses in an ECG and eliminate false detections due to muscle artifact, noise spikes, electrode artifact and narrow QRS complexes. Analysis can also provide an accurate and meaningful classification and interpretation describing the observed pacemaker's interaction with the cardiac rhythm.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIGS. 1A-C are a series of plots illustrating the effect a pacemaker can have on an ECG signal;

FIG. 2A-B are two plots illustrating, the relatively consistent morphology of a pace pulse for a patient when compared to the variable morphology of noise signals measured;

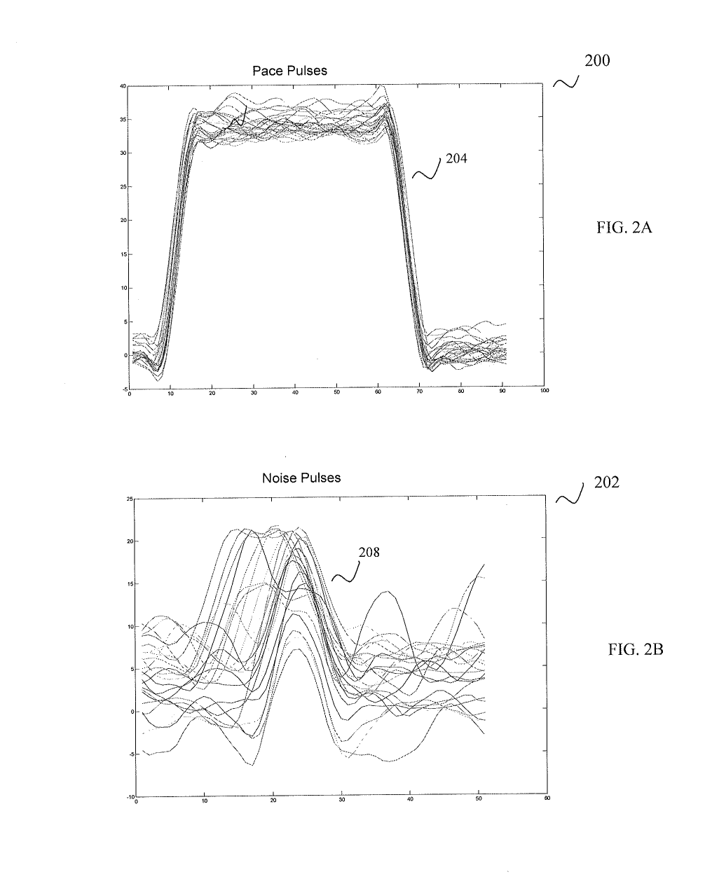

FIG. 3 is a process flow diagram illustrating detection of a pace pulse from ECG data;

FIG. 4 is a series of plots illustrating two example pace pulse and template comparisons;

FIG. 5 is a schematic diagram illustrating a system for detecting pace pulses in ECG signals; and

FIG. 6 is a series of plots illustrating an ECG signal measured on four different leads.

### DETAILED DESCRIPTION

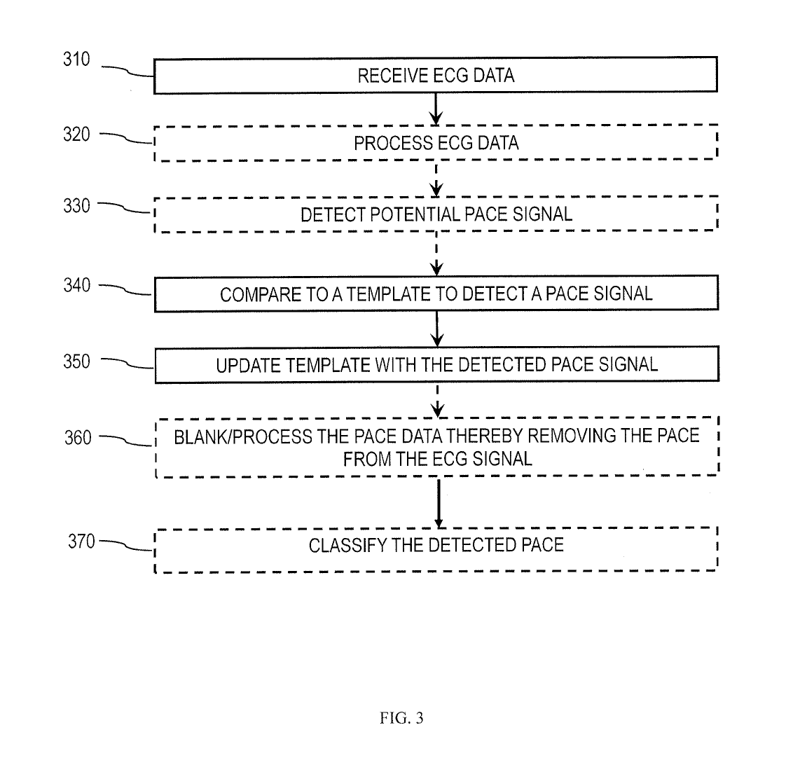

FIG. 3 is a process flow diagram 300 illustrating a process of detecting a pace pulse from ECG data. At 310, data characterizing ECG measurements are received. The data can include measurements from any number of ECG leads sampled by an analogue to digital converter (A/D).

Optionally, at step 320, the ECG data can be processed. This can include steps such as filtering, smoothing, up or down sampling, etc. Optionally, at step 330, a portion of the ECG data is selected as a potential pace pulse. This selection can be based on drastic and quick changes in ECG signal value that can be indicative of a pace pulse, e.g. a “spike” in the ECG signal.

At step 340, at least a portion of the ECG data is compared to at least one template. If applicable (i.e. step 330 was performed), the portion of ECG data can be the potential pace pulse. Each template can comprise one or more previously detected pace signals or a composition thereof. The comparison can include a determination as to the similarity of the portion of the ECG data to each template and if the portion of the ECG data is sufficiently similar to at least one template, the pace pulse is detected. There can be three or more templates. The templates can correspond to an atrial pace pulse, a right ventricular pace pulse, and a left ventricular pace pulse. Other templates are possible.

An example measure of similarity is the Pearson product-moment coefficient. The Pearson product-moment coefficient assessed the degree of similarity between two variables X and Y using the following equation:

$$r = \frac{{\sum }_{i = 1}^{n} ( {X}_{i} - \overline{X} ) ( {Y}_{i} - \overline{Y} )}{\sqrt{{\sum }_{i = 1}^{n} {( {X}_{i} - \overline{X} )}^{2}} \sqrt{{\sum }_{i = 1}^{n} {( {Y}_{i} - \overline{Y} )}^{2}}} .$$

Other example measures of similarity can include: kendall tau correlation, spearman's rank correlation, co-variance, sample correlation, etc.

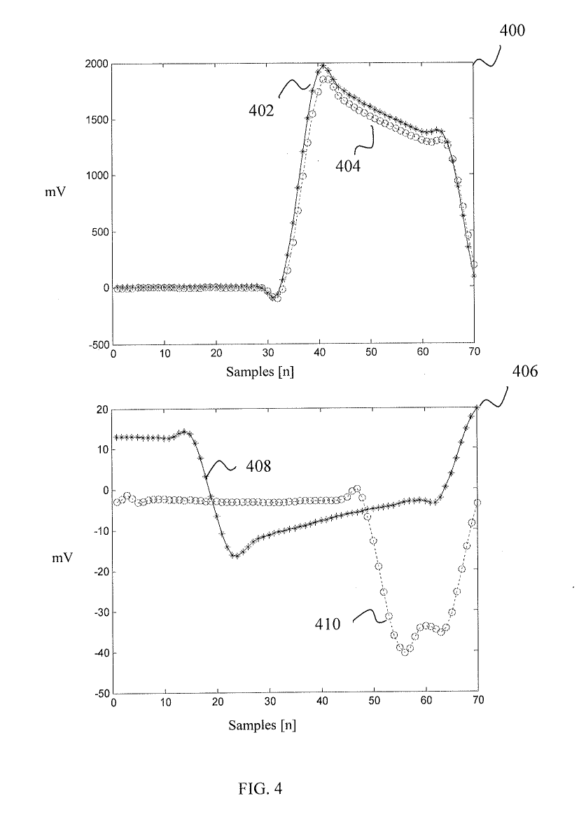

FIG. 4 is a series of plots illustrating two example pace pulse and template comparisons. In plot 400, a seventy sample window of a template pulse 402 created from previously detected similar pace pulses is compared to a seventy sample window of a portion of ECG data (new detected pace pulse) 404. The seventy samples are measured over 2.2 milliseconds. The resulting coefficient value is 0.9974 indicating a high similarity. Plot 406 illustrates a template pulse 408 compared to a non-matching portion of ECG data 410. The resulting coefficient value is 0.0463 indicating a low similarity. The coefficient can be compared to a predetermined threshold to determine if a pace pulse is present in the ECG data.

Another example measure of similarity is an amplitude comparison. Amplitudes can be considered similar if the template pulse amplitude is within a range of the ECG data amplitude. For example, amplitudes can be considered similar if the template pulse amplitude is within 25% of the portion of ECG data amplitude.

Returning to FIG. 3, at step 350 the template is updated based in part on the matched pace pulse. The template can be updated, for example, based on the following: New_Template=0.85*Old_Template+0.15*Matched_Pace_Pulse. The updating will ensure that the template reflects the current pace pulse morphology in cases where the morphology varies slightly over time. For example, the morphology could vary over time in a clinical setting due to patient movement, ECG electrode drying, slight ECG electrode re-positioning or movement, pacemaker lead movement, the pacemaker adjusts the pace pulse amplitude slightly, etc. The template can be dynamic.

Optionally, at step 360, using the detected pace pulse, the portion of the ECG signal can be blanked thereby removing the pace pulse from the ECG signal. The pace pulse can be replaced (i.e. blanked) by interpolating between the non-removed ECG data points. The portion of the ECD data which is removed is the blanking period. Further, the pace pulse tail may also be removed by blanking.

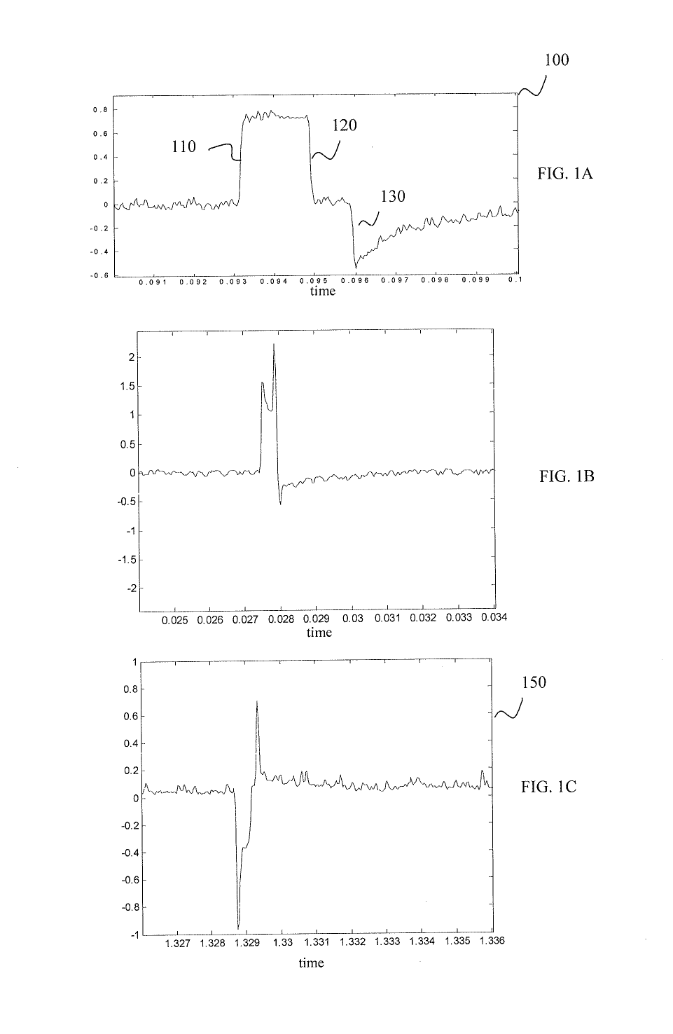

A pace pulse consists of the main pulse and the repolarization pulse (i.e. pace pulse tail, see FIG. 1, label 130). To remove the entire pace pulse artifact from the ECG data both the main pulse and tail can be removed. The main pulse corresponds to the energy delivered to a heart to stimulate the cardiac muscle and cause it to contract. The pace pulse tail corresponds to the depletion of the capacitive coupling generated by the delivery of the main pulse (i.e. the depletion of the potential built up between the heart and the pacemaker). The amount of energy that is depleted is the same as the amount of energy delivered. Further, the pace pulse tail can be described as having a shape of an exponentially decaying signal, (and can be described similar to the electrical behavior of a resistor-capacitor circuit). Therefore, after detecting the main pulse, the amplitude and width of the pace pulse can be used to determine the energy delivered in the pace pulse and be used to determine an expected width of the pace tail. Given an estimated pace tail width, a determination can be made as to an appropriate duration of the blanking period required to sufficiently blank both the main pace pulse and the pace pulse tail.

Optionally, at step 370, the pace pulse can be classified. The pace pulse can be classified an atrial pace pulse, right ventricular pace pulse, or left ventricular pace pulse. The pace pulse can be classified based on the template the pace pulse shape is most similar to.

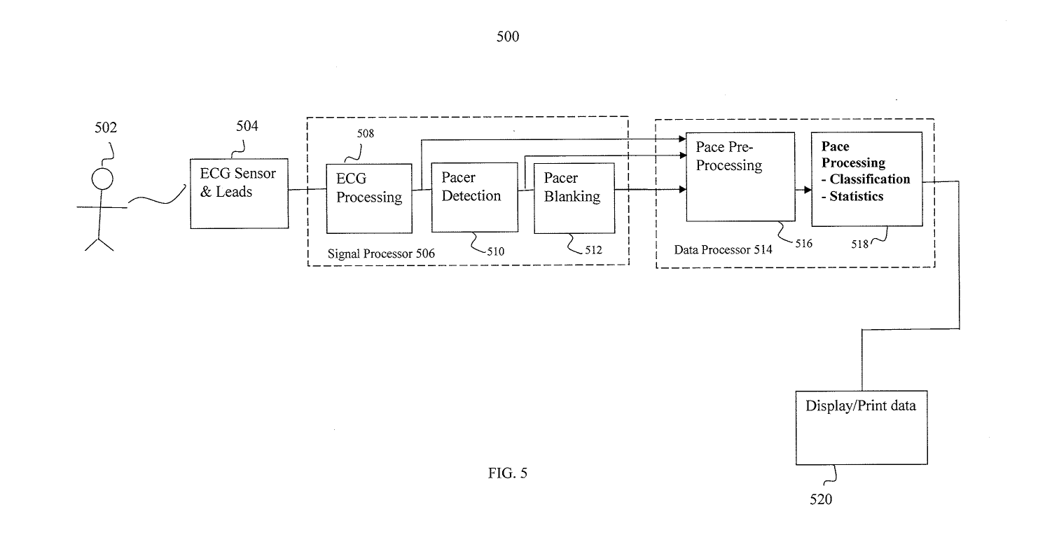

FIG. 5 is a schematic diagram 500 illustrating a system for detecting pace pulses in ECG signals. A patient 502 can be connected to an ECG sensor 504. The ECG sensor can comprise any number of leads, such as 3-lead, 5-lead or 12-lead. The ECG measurements can be passed to a signal processor 506. The signal processor can include an ECG processor 508, a pacer detector 510, and a pace blanker 512. The ECG processor 508 can include an A/D converter, filters, and can perform processing such as up or down sampling. The pacer detector 510 can receive ECG data from the ECG processor 508 and can compare the data to at least one template pulse. The template pulse can be created from previously detected similar pace pulses. The pacer detector can compute a similarity between at least a portion of the ECG data and at least one template to detect the presence of a pace pulse in the ECG data. The pace blanker 512 can remove the detected pace pulse from the ECG data and can interpolate the ECG data to replace the pace pulse.

The ECG data from the ECG processor 508, the detected pace pulse from the pacer detector 510 and the blanked ECG data from the pace blanker 512 can be passed to a data processor 514. The data processor can include a pace preprocessor 516 for checking for pacer activity and a pace processor 518 for classifying the pace pulses and aggregating statistics over time relating to the pace pulse types, timing, and other characteristics. The data processor can be connected to a display and/or printer 520 for viewing data relating to the ECG measurements and pace pulse detections.

Each ECG lead can provide a different view of the heart and pacing can occur from various pacemaker lead positions and across various pacing vectors depending on the patient and pacemaker/ICD/lead system. Therefore each situation is unique and there may be no single ECG lead that will be best for all patients. Therefore, detecting pace pulses on multiple ECG leads and cross checking pace detection can offer additional detection robustness and performance.

For example, pace detection can be performed on 4 ECG leads and then leads can be disqualified based on signal quality. Individual ECG lead pace pulse detections can be grouped together if they occur within, e.g., 3 milliseconds of each other. If signal quality is good on all 4 leads than a pace pulse can be detected based on a voting scheme, such as if it is detected on 2 of the 4 leads. If signal quality is good on only 2 or 3 leads than the best and second best lead in terms of signal quality can be logically OR'd before determining if a pace pulse is detected. If signal quality is only good on 1 lead or no leads than the lead with the best signal quality should be used for pace detection.

Signal quality can be assessed based on the signal to noise ratio and it can be reassessed for each pace pulse. The noise level can be assessed over the samples before the pace pulse detection. The maximum noise amplitude can be assessed as the difference between the max value and min value in the samples before the pace pulse detection. The noise level used for the signal to noise ratio calculation can be, for example, 0.125*current max noise amplitude+(1-0.125)*previous max noise amplitude, or any other measure of noise (such as the mean, median noise amplitude, etc.). The signal level can be the detected pace pulse peak amplitude, if no pace pulse was detected on that lead the signal level can be an average of the 6 previously detected pace pulses. The signal to noise ratio can be compared to a threshold to be considered a valid lead. For example, a signal to noise ratio can be greater than 2 to be considered a valid lead.

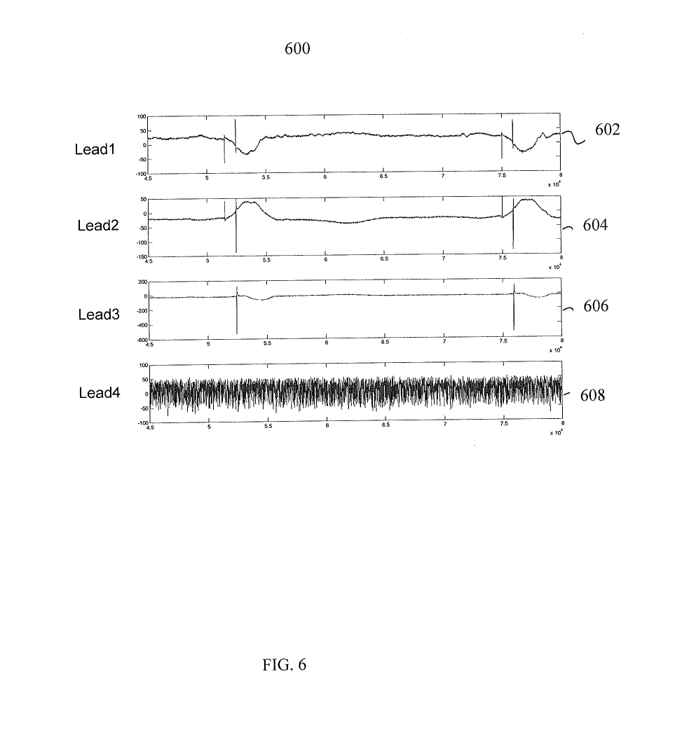

FIG. 6 is a series of plots illustrating an ECG signal measured on four different leads. Plots 602, 604, and 606 show data from valid leads. For the first ventricular pace pulse, the lead shown on 602 and the lead shown on 604 have the best signal quality and therefore can be used for detection. For the second ventricular pace pulse the lead shown on 604 and the lead shown on 606 have the best signal quality and therefore can be used for detection. The lead shown on 608 is invalid and therefore may not be used for detection.

To initiate a template, there can be a predetermined number of empty potential template slots. For a given portion of ECG data, if the data does not match any existing template, it can become a potential template and fill a potential template slot. Each time a potential template matches a new pulse, a match count for that template can be increased. After a predetermined number of matches, the potential template will become a template.

Once templates have been initiated, it can be necessary to clear one or all templates if, for example, a noise pulse mistakenly becomes a template or if the pacemaker/ICD is re-programmed and a template is no longer applicable. Therefore, if the interval between pace pulses is less than the pacemaker is configured to pace, such as 150 beats per minutes for an adult or 180 beats per minute for a pediatric, a “clear count” can be incremented for the template that matches the pace pulses. When the “clear count” exceeds a predetermined threshold the template can be cleared.

Various implementations of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user and a keyboard and a pointing device (e.g., a mouse or a trackball) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

Although a few variations have been described in detail above, other modifications are possible. For example, the logic flow depicted in the accompanying figures and described herein do not require the particular order shown, or sequential order, to achieve desirable results. Other embodiments may be within the scope of the following claims.

1.-25. (canceled)

26. A method for implementation by one or more data processors forming part of at least one computing system, the method comprising:
receiving, by at least one data processor, data characterizing an electrocardiogram (ECG) measured from a patient having a pacemaker intermittently providing a pace to a heart of the patient;
comparing, using at least one data processor, at least a portion of the received data to a template by correlating the at least a portion of the received data to the template thereby detecting a pace pulse from at least a portion of the received data, the template comprising one or more pace pulses measured from the patient; and
providing, using at least one data processor, data characterizing the detected pace pulse.

27. The method of claim 26, further comprising:
blanking the received data for a blanking period thereby removing the detected pace pulse from the received data.

28. The method of claim 27, wherein the length of the blanking period is determined by at least one characteristic of the detected pace pulse, wherein the at least one characteristic is one or more of: pace pulse width and pace pulse amplitude.

29. The method of claim 26, further comprising:
classifying the detected pace pulse.

30. The method of claim 29, wherein the detected pace pulse is classified as a pulse selected from a group consisting of: atrial pace pulse, right ventricular pace pulse or left ventricular pace pulse.

31. The method of claim 29, further comprising:
calculating the occurrence of the classified detected pace pulse.

32. The method of claim 26, wherein the received data characterizing ECG data measured from a patient has been processed by removing baseline variation using a filter.

33. The method of claim 26, wherein the template is modified, at least in part, by the detected pace pulse to update the template.

34. The method of claim 26, wherein the pace pulse is detected when the amplitudes of the at least a portion of received data and the template differ by less than about 25%.

35. The method of claim 26, wherein the comparison of the at least a portion of the received data to the template is calculated according to:
$$r = \frac{{\sum }_{i = 1}^{n} ( {X}_{i} - \overline{X} ) ( {Y}_{i} - \overline{Y} )}{\sqrt{{\sum }_{i = 1}^{n} {( {X}_{i} - \overline{X} )}^{2}} \sqrt{{\sum }_{i = 1}^{n} {( {Y}_{i} - \overline{Y} )}^{2}}} .$$

36. The method of claim 26, wherein the received data includes data from four or more leads and wherein the at least a portion of received data corresponds to a subset of the ECG leads having the highest signal to noise ratio when compared to all leads.

37. The method of claim 26, wherein providing the data comprises one or more of persisting the data, displaying the data, or transmitting the data to a remote module or computing system.

38. The method of claim 26, wherein the template is a dynamic template.

39. A method for implementation by one or more data processors forming part of at least one computing system, the method comprising:
receiving, by at least one data processor, electrocardiogram (ECG) data measured from a patient having a pacemaker intermittently providing a pace to a heart of the patient;
selecting, using at least one data processor and from at least a portion of the received data, potential pace data;
comparing, using at least one data processor, the potential pace data to at least one template to determine if the potential pace data is the result of the heart being paced by the pacemaker thereby detecting the pace pulse, the template being determined from one or more previously detected paces measured from the patient;
providing, using at least one data processor, data characterizing the detected pace pulse; and
blanking, using at least one data processor, the received data for a period thereby removing the potential pace data from the received data.

40. The method of claim 39, further comprising:
modifying, using at least one data processor, the template by at least the potential pace data to create a modified template.

41. The method of claim 39, wherein the length of blanking period is determined by at least one characteristic of the potential pace data, wherein the at least one characteristic is selected from the group comprising: pace width and pace amplitude.

42. The method of claim 39, further comprising:
classifying the detected pace data, wherein the detected pace signal is classified as a signal selected from a group consisting of: atrial pace pulse, right ventricular pace pulse or left ventricular pace pulse.

43. The method of claim 29, wherein the received data characterizing ECG data measured from a patient has been processed.

44. The method of claim 43, wherein the received data is processed by removing baseline variation using a filter.

45. A system comprising:
an electrocardiogram (ECG) sensor including a plurality of ECG signal path leads configured to be placed on a patient having a pacemaker intermittently providing a pace to a heart of the patient; and
a signal processor configured to receive the ECG signal from the ECG sensor, compare at least a portion of the ECG signal to one or more templates thereby detecting the presence of a pace signal indicating the heart has been paced by the pacemaker and provide data characterizing the detected pace pulse;
wherein the signal processor is further configured to blank the ECG signal thereby removing the detected pace signal from the ECG signal and configured to update at least one of the one or more templates based, at least in part, on the detected pace signal.

46. The system of claim 45, further comprising:
at least one data processor couple to memory storing instructions which, when executed by the at least one data processor, causes the at least one data processor to perform operations comprising:
receiving data characterizing the detected pace signal; and
classifying the received data.

47. The system of claim 45, wherein the degree of blanking is determined by at least one characteristic of the detected pace signal, wherein the at least one characteristic is selected from the group comprising: pace signal width and pace signal amplitude.

48. The system of claim 45, wherein the comparing of at least a portion of the ECG signal to the one or more templates includes determining a correlation between the at least a portion of ECG signal and each template.
